(19) United States
(12) Patent Application Publication
(10) Pub. No.: US 2008/0233162 A1
(43) Pub. Date: Sep. 25, 2008

(54) Fibrous 3-Dimensional Scaffold Via Electrospinning For Tissue Regeneration and Method For Preparing the Same

(76) Inventors: Seung Jin Lee, Seoul (KR); Sol Han, Seoul (KR); In Kyong Shim, Gyeonggi-do (KR)

Correspondence Address: JHK LAW, P.O. BOX 1078, LA CANADA, CA, 91012-1078, US

(21) Appl. No.: 12/064,801

(22) PCT Filed: Aug. 28, 2006
(86) PCT No.: PCT/KR2006/003390

§ 371 Date: Feb. 25, 2008

(30) Foreign Application Priority Data
Aug. 26, 2005 (KR) 10-2005-0078640

(51) Int. Cl. A61K 9/00; C12N 5/06; A61K 35/12; A61P 43/00

(52) U.S. Cl. 424/422; 435/396; 424/93.7

(57) ABSTRACT

The present invention relates to a fibrous 3-dimensional porous scaffold via electrospinning for tissue regeneration and a method for preparing the same. The fibrous porous scaffold for tissue regeneration of the present invention characteristically has a biomimetic structure established by using electrospinning which is efficient without wasting materials and simple in handling techniques. The fibrous porous scaffold for tissue regeneration of the present invention has the size of between nanofiber and microfiber and regular form and strength, so that it facilitates 3-dimensional tissue regeneration and improves porosity at the same time with making the surface area contacting to a cell large. Therefore, the scaffold of the invention can be effectively used as a support for the cell adhesion, growth and regeneration.

### TECHNICAL FIELD

The present invention relates to a fibrous 3-dimensional porous scaffold via electrospinning for tissue regeneration and a method for preparing the same.

### BACKGROUND ART

Tissue regeneration is induced by supplying cells or drug loaded matrix when tissues or organs lose their functions or are damaged. At this time, a scaffold for tissue regeneration has to be physically stable in the implanted site, has to be physiologically active to control regeneration efficacy, has to be easily degraded in vivo after generating new tissues and must not produce degradation products with toxicity.

The conventional scaffolds for tissue regeneration have been produced by using polymers having a certain strength and form, for example sponge type or fibrous matrix or gel type cell culture scaffold has been used.

The conventional fibrous matrix scaffold has open cellular pores and the pore size is enough size that cells are easily adhered and proliferated. However, the fibrous matrix scaffold is not commonly used today as its disadvantages have been confirmed as follows; a scaffold composed of natural polymer has so poor strength in water phase that it might be destroyed or contracted to lose its original form, and even a synthetic polymer scaffold cannot secure a room with its fibrous structure alone, so that it ends in the membrane shaped 2-dimensional structure rather than 3-dimensional structure. The 3-dimensional structure is very important for tissue regeneration and activity. So, such scaffolds having only 2-dimensional structure are limited in applications since it is very difficult with these scaffolds to envelop a medicine and regulate its release or to employ a natural polymer with high physiological activity.

The preparing method of a sponge type scaffold has been generally accepted for the preparation of conventional scaffolds for tissue generation, for example, particle leaching, emulsion freeze-drying, high pressure gas expansion and phase separation, etc.

The particle leaching technique is that particles which are insoluble in bio-degradable polymer with organic solvent such as salt are mixed with a casting, a solvent is evapotated and then the salt particles are eliminated by elution in water. According to this method, a porous structure with cellular pores in different sizes and various porosities can be obtained by regulating the size of the salt particle and the mixing ratio. However, it is a problem of this method that the remaining salts or rough surfaces cause cell damage (Mikos et al., Biomaterials, 14: 323-330, 1993; Mikos et al., Polymer, 35: 1068-1077, 1994).

Emulsion freeze-drying is the method that the emulsion of a polymer with organic solvent and water is freeze-dried to eliminate the residual solvents. In the meantime, high pressure gas expansion method does not use any organic solvent. According to this method, a bio-degradable polymer is introduced into a mold and pressure is given thereto to prepare pellet. Then, high pressure carbon dioxide is injected into the bio-degradable polymer at a proper temperature and then the pressure is reduced to release carbon dioxide in the mold to form cellular pores. However, the above methods are also limited in producing open cellular pores (Wang et al., Polymer, 36: 837-842, 1995; Mooney et al., Biomaterials, 17: 1417-1422, 1996).

Another attempt has recently been made to prepare porous scaffold based on phase separation. Particularly, a sublimable substance or another solvent having different solubility is added to a polymer organic solvent and then phase separation of the solution is performed by sublimation or temperature change. However, this method has also a problem of difficulty in cell culture because the size of the produced pore is too small (Lo et al., Tissue Eng. 1: 15-28, 1995; Lo et al., J. Biomed. Master. Res. 30: 475-484, 1996; Hugens et al., J. Biomed. Master. Res., 30: 449-461, 1996).

The above mentioned methods are to prepare a 3-dimensional polymer scaffold which is capable of inducing cell adhesion and differentiation, but using a bio-degradable polymer for the production of a 3-dimensional scaffold for tissue re-generation has still a lot of problems to be overcome.

A polymer scaffold prepared by using electrospinning has been evaluated, but re-sultingly confirmed that it ends up in 2-dimensional membrane structure, which means it is very difficult to use this scaffold as a 3-dimensional structured implantation material with successful cell adhesion (Yang et al., J. Biomater. Sci. Polymer Edn., 5:1483-1479, 2004; Yang et al., Biomaterials, 26: 2603-2610, 2005).

An extracellular matrix in vivo has a network-structure composed of basic materials such as glycosaminoglycan and collagen nanofiber, in which cells are adhered and pro-liferated to form tissues.

To overcome the problems of the conventional polymer scaffold for tissue re-generation, the present inventors paid attention to the extracellular matrix like structure and finally completed this invention by producing, for the first time in Korea, a fibrous 3-dimensional polymer scaffold which has structural similarity with the extracellular matrix, regular form and strength and the size of between nanofiber and microfiber so that it enables successful 3-dimensional tissue regeneration.

### DISCLOSURE OF THE INVENTION

### Technical Problem

It is an object of the present invention to provide a 3-dimensional polymer scaffold for tissue regeneration having the size of between nanofiber and microfiber to provide large surface for cell adhesion and thus forming a 3-dimensional structure for successful tissue regeneration.

### Technical Solution

To achieve the above object, the present invention provides a fibrous porous 3-dimensional scaffold for tissue regeneration comprising a polymer fiber having a 3-dimensional network structure using electrospinning.

The present invention also provides a method for preparing the fibrous porous 3-dimensional scaffold for tissue regeneration using electrospinning.

Hereinafter, the present invention is described in detail.

The present invention provides a fibrous porous 3-dimensional scaffold for tissue regeneration having a 3-dimensional network structure comprising a polymer fiber having the size of between nanofiber and microfiber.

FIGS. 2, 3 and 4 illustrate examples of the fibrous porous scaffolds of the invention which are 3-12 [symbol] in diameter, which is the size of between nanofiber (1-500 nm) and microfiber (30-50 [symbol] ). The scaffold of the invention has as small fiber diameter as possible to provide large surface area for successful cell adhesion and proliferation and at the same time a regular form and strength to enhance 3-dimensional tissue re-generation capacity.

The fibrous porous scaffold of the present invention contains a bio-degradable polymer composed of one or more natural polymers selected from a group consisting of chitosan, chitin, alginic acid, collagen, gelatin and hyaluronic acid and a bio-degradable polymer composed of a representative bio-degradable aliphatic polyester selected from a group consisting of polylactic acid (PLA), polyglycolic acid (PGA), poly(D,L-lactide-co-glycolide) (PLGA), poly(caprolactone), diol/diacid aliphatic polyester and polyester-amide/polyester-urethane and one or more synthetic polymers selected from a group consisting of poly(valerolactone), poly(hydroxyl butyrate) and poly(hydroxyl valerate).

The synthetic polymer is preferably polylactic acid (PLA) having the molecular weight of 100,000-350,000 kD, but not always limited thereto. The synthetic polymer is more preferably poly L-lactic acid (PLLA).

Either a natural polymer or a synthetic polymer can be used alone or both of them can be used at the same time as a mixture.

The fibrous porous scaffold of the present invention has the size of between nanofiber and microfiber, preferably 1-15 < in diameter, and a regular form and strength under a proper pressure to help 3-dimensional tissue regeneration and at the same time to provide a large surface area for cell adhesion, so that it can be effectively used for adhesion and proliferation of such cells as endothelial cells, skin cells and osteocytes. In addition, the scaffold of the invention can be simply prepared by using electrospinning without wasting of polymers or drugs, so it can be more efficient than any other method.

The fibrous porous scaffold of the present invention can include not only a polymer but also a synthetic low molecular compound.

The present invention also provides a method for preparing the porous fibrous scaffold with polymer.

Particularly, the present invention provides a method for preparing the fibrous porous scaffold comprising the following steps:

(i) preparing a spinning solution by dissolving a polymer and a low-molecular compound singly or together in an organic solvent; and

(ii) spinning the polymer solution by using an electro-spinner and volatilizing the organic solvent at the same time to form a 3-dimensional network structure; and at last molding the produced fiber having the size of between nanofiber and microfiber to fit defective area.

In the above step (i), to prepare the spinning solution, a natural polymer or a synthetic polymer is dissolved in an organic solvent singly or together and a drug is additionally dissolved therein. In step (i), poly L-lactic acid (PLLA) was dissolved in the organic solvent.

Any volatile organic solvent having a low boiling point can be used as an organic solvent for the invention to dissolve the synthetic polymer above and particularly chloroform, dichloromethane, dimethylformamide, dioxane, acetone, tetrahydrofurane, trifluoroethane and 1,1,1,3,3,3,-hexafluoroisopropylpropanol are preferred and dichloromethane is more preferred but not always limited thereto.

According to the present invention, the polymer solution drips on a collector by electrospinning and at this time the solvent is entirely volatilized. Because of electrostatic repulsive power, there is no direct contact between fiber and fiber, indicating that fibers are integrated separately. What is most important in this process is that all the solvent has to be volatilized before the drip of the polymer solution on the collector, for which the boiling point of the solvent has to be very low and viscosity of the solvent has to be properly adjusted. Particularly, the preferable boiling point and viscosity of the solvent is 0-40° C. and 25-35 cps respectively. It is also important to maintain a proper temperature and humidity.

A polymer and a low molecular compound included in the fibrous 3-dimensional polymer scaffold are dissolved in 5-20 weight % of an organic solvent to prepare a spinning solution.

According to the method for preparing the porous 3-dimensional scaffold of the invention, when temperature, humidity, viscosity of the solution and volatility of the solvent are optimized, fibers are not directly adhered and integrated separately, simply resulting in the 3-dimensional scaffold by itself.

In step (ii), a fiber is prepared by using the spinning solution with electro-spinner.

The spinning process by electro-spinner is described in detail hereinafter (see FIG. 1).

Electric field is formed between nozzle and collector by applying a certain current from voltage generator. The polymer solution filled in the spinning solution depository is spun on the collector by the force of the electric field and the pressure from syringe pump. At this time, voltage, flowing speed, the electric field distance between nozzle and collector, temperature and humidity are important factors affecting spinning. In particular, the concentration of the spinning solution affects the diameter of a fiber most significantly. So, all the conditions of the electro-spinner are optimized to prepare a fiber of the invention.

The conditions of the electro-spinner are as follows; spinning distance: 10-20 cm, voltage: 10-20 kV and spinning speed: 0.050-0.150 ml/min, but not always limited thereto. The electro-spinner used in the present invention is DH High Voltage Generator (CPS-40KO3VIT, Chungpa EMT, Korea).

The present invention further provides an implantation material for cell adhesion, growth and regeneration containing the fibrous porous 3-dimensional scaffold for tissue regeneration of the invention. The applicable cells are not limited but cartilage cells, endothelial cells, skin cells, osteocytes, bone cells and stem cells are preferred.

### BRIEF DESCRIPTION OF THE DRAWINGS

The application of the preferred embodiments of the present invention is best understood with reference to the accompanying drawings, wherein:

FIG. 1 is a schematic diagram illustrating the spinning using an electro-spinner.

FIG. 2 is a photomicrograph (X 500) of fiber prepared under the conditions of double electric field length: 20 cm, voltage: 10 V, release rate: 0.060 ml/min., and inner diameter of needle: 1.2 mm.

FIG. 3 is a photomicrograph (X 3500) of fiber prepared under the conditions of double electric field length: 20 cm, voltage: 10 V, release rate: 0.060 ml/min., and inner diameter of needle: 1.2 mm.

FIG. 4 is a photomicrograph (X 2000) showing the surface of the fibrous porous scaffold prepared by electrospinning under the conditions of double electric field length: 20 cm, voltage: 10 V, release rate: 0.060 ml/min., and inner diameter of needle: 1.2 mm.

FIG. 5 is a photomicrograph(X 2000) showing osteoblasts cultured for 7 days in low molecular scaffold.

FIG. 6 is a set of photomicrograph(X 500) showing osteoblasts cultured for 14 days in low molecular scaffold.

FIG. 7 is appearance of electrospun PLLA sub-micro fibrous scaffold. (A) electrospun fibers, (B) 3-D formed scaffold after handling electrospun fibers.

### MODE FOR THE INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Preparation of a Polymer PLLA Fiber

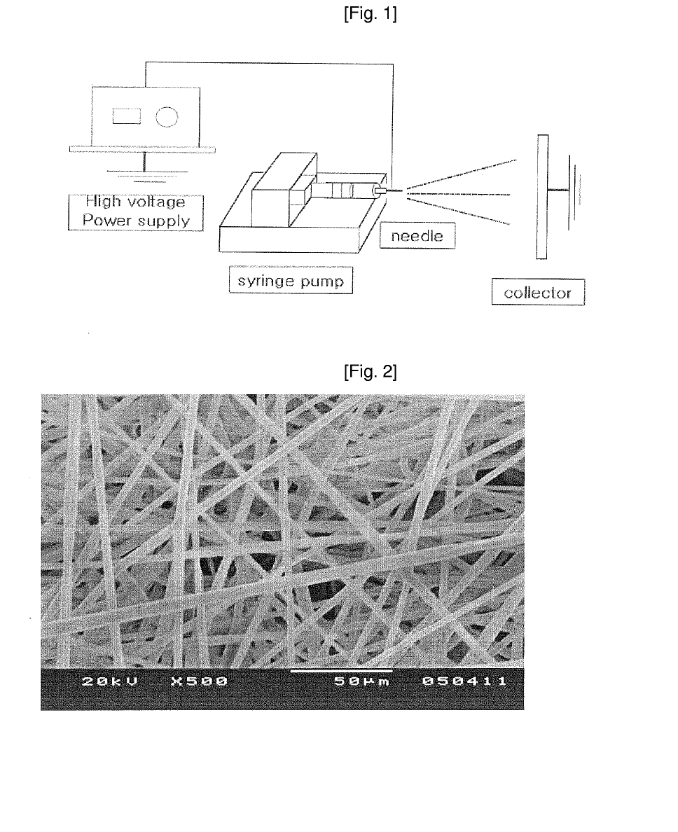

A PLLA polymer was dissolved in 10 < of dichloromethane solution, resulting in a 5-10% spinning solution. A fiber was prepared from the spinning solution by electrospinning (FIG. 1).

As an electro-spinner, DH High Voltage Generator (CPS-40KO3VIT, Chungpa EMT, Korea) was used and the details of the electrospinning process are illustrated with the reference to FIG. 1.

The 5-10% polymer PLLA solution (spinning solution) was filled in a spinning solution depository, which was a 10 < glass syringe. A needle with blunt tip, which is 0.5-1.2 mm in diameter, was used. The releasing speed of the spinning solution was adjusted to 0.060 ml/min. Voltage was set at 10-20 kV and the electric field distance was adjusted to 10-20 cm. It was important for the entire solvent to be volatilized before the drip of the solution on a collector to prepare a target fiber. Thus, the temperature and humidity had to be carefully regulated; the optimum temperature was 15-20° C. and the optimum humidity was 10-40%.

The prepared polymer PLLA fiber was confirmed to be 3-10 < in thickness.

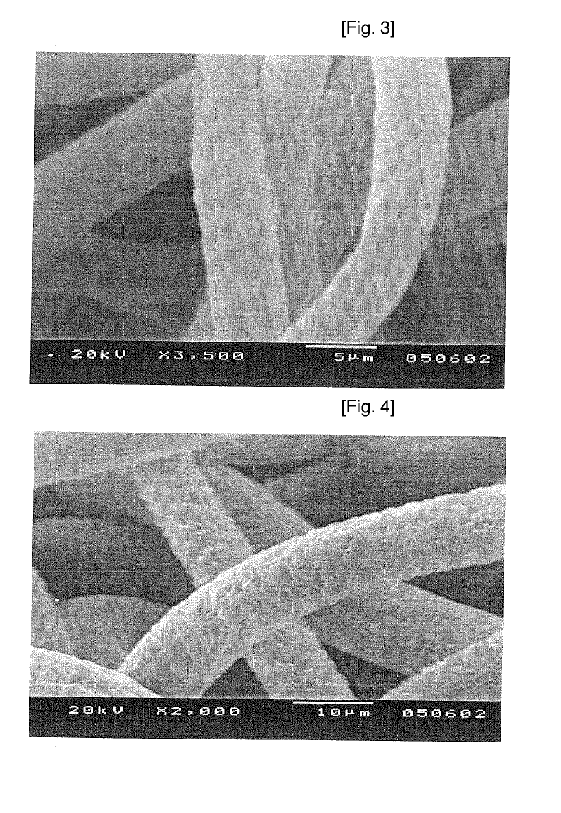

FIGS. 2 and 3 are photomicrographs (X 500, X 3500) of fibers prepared under the conditions of 20 cm of double electric field distance, 10 V of voltage, 0.060 ml/min of releasing speed and 1.2 mm of the internal diameter of a needle.

### EXAMPLE 2

### Preparation of a Low Molecular PLLA Fiber

A low molecular PLLA was dissolved in 10 < of dichloromethane solution, resulting in a 14-20% spinning solution. A fiber was prepared from the spinning solution by electrospinning (FIG. 1).

As an electro-spinner, DH High Voltage Generator (CPS-40KO3VIT, Chungpa EMT, Korea) was used and the details of the electrospinning process are illustrated with the reference to FIG. 1.

The 14-20% low molecular PLLA solution (spinning solution) was filled in a spinning solution depository, which was a 10 < glass syringe. A needle, which is 0.5-1.2 mm in diameter, was used. The releasing speed of the spinning solution was adjusted to 0.060 ml/min. Voltage was set at 10-20 kV and the electric field distance was adjusted to 10-20 cm. It was important for the entire solvent to be volatilized before the drip of the solution on a collector to prepare a target fiber. Thus, the temperature and humidity had to be carefully regulated; the optimum temperature was 15-25° C. and the optimum humidity was 10-40%.

The prepared low molecular PLLA fiber was confirmed to be 5-10 < in thickness.

FIG. 2 is a photomicrograph (X 2000) of a fiber prepared under the conditions of 10 cm of double electric field distance, 10 V of voltage, 0.060 ml/min of releasing speed and 1.2 mm of the internal diameter of a needle.

### EXAMPLE 3

### Preparation of a Spinning Solution using Dichloromethane and 1,1,1,3,3,3-hexafluoroisopropylpropanol

To dichloromethane was added 1,1,1,3,3,3-hexafluoroisopropylpropanol by 2% of the total solvent, resulting in dichloromethane solution. Then, polymer and low molecular PLLA were dissolved in the dichloromethane solution to prepare a spinning solution with proper concentrations of the polymer and low molecular PLLA. A fiber was prepared from the spinning solution by electrospinning. The resultant fiber was proved to be very stable in shape and spun at a wide range of temperature and humidity (possibly spun even at 30° C. with 50% humidity). The obtained polymer was confirmed to be 1-10 < in diameter. The addition of 1,1,1,3,3,3-hexafluoroisopropylpropanol caused the fiber to be thinner and more stable spinning, but at the same time, increased electrostatic force between fibers and formed a shield-like membrane.

### EXAMPLE 4

### Preparation of a Spinning Solution using Dichloromethane and Acetone

To dichloromethane was added acetone by 10% of the total solvent, resulting in dichloromethane solution. Then, polymer and low molecular PLLA were dissolved in the dichloromethane solution to prepare a spinning solution with proper concentrations of the polymer and low molecular PLLA. A fiber was prepared from the spinning solution by electrospinning. The resultant fiber was proved to be very stable in shape and spun at a wide range of temperature and humidity (possibly spun even at 30° C. with 50% humidity). However, no changes in diameter were observed. The addition of acetone results in the same fiber as obtained by using dichloromethane alone and stabilized the spinning better, suggesting that the added acetone could supplement sensitive factors to enhance the efficiency.

### EXAMPLE 5

### Osteoblasts Adhesion Test

The following experiment was performed to investigate the adhesion capacity of the porous scaffold of the present invention.

The fibrous scaffolds prepared in Examples 1 and 2 were sterilized with 70% ethanol, on which sub-cultured osteoblasts (MC3TC) were static cultured. Observation on the adhered cells was performed under differential scanning microscope.

The cells remaining without being adhered were eliminated. 25% (w/w) glutaraldehyde was diluted in 0.1 M phosphate buffered saline (PBS, pH 7.4), resulting in 2.5% glutaraldehyde solution, with which pre-fixation was carried out for 4-20 minutes. After the fixation, water was eliminated by using ethanol, followed by freeze-drying. Then, the sample was coated with gold and observed under differential scanning microscope.

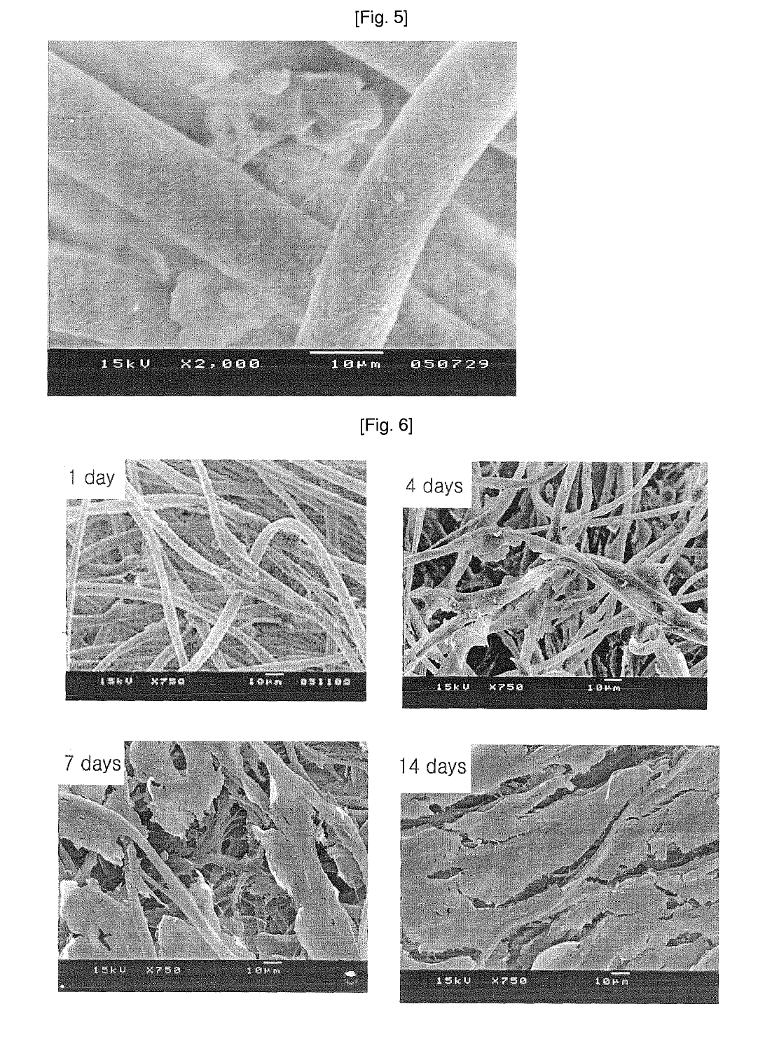

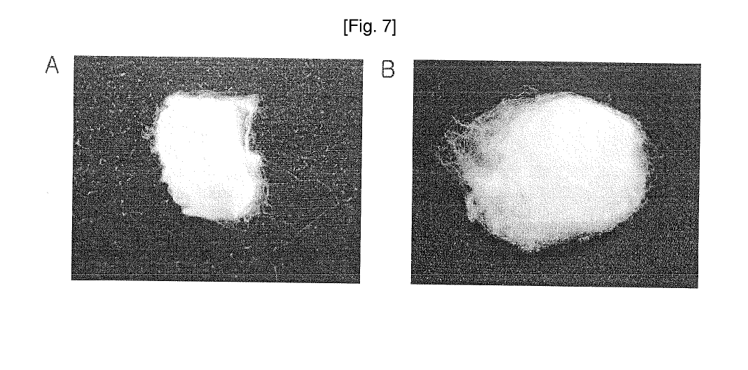

As a result, the prepared fiber was still stable in shape and in strength even after 7 days from the preparation and osteoblasts were packed between and on the surfaces of the fibers. Accordingly, it was confirmed that the porous scaffold of the present invention had cellular affinity, so that cells could be adhered stably. Therefore, the porous scaffold of the invention can be accepted as an appropriate scaffold material (FIGS. 5, 6 and 7).

### INDUSTRIAL APPLICABILITY

The fibrous porous scaffold for tissue regeneration of the present invention has a biomimetic structure, which can be prepared by using electrospinning efficiently and with simple techniques. The fibrous porous scaffold for tissue regeneration of the invention has the size of between nanofiber and microfiber and a regular form and strength, so that it enables 3-dimensional regeneration of biological tissues and enhances porosity, suggesting that the cell-contacting surface area becomes large to facilitate cell adhesion, growth and regeneration.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A fibrous porous 3-dimensional scaffold for tissue regeneration comprising a polymer and/or a low molecular fiber, which is formed in a 3-dimensional network structure by electrospinning.

2. The fibrous porous 3-dimensional scaffold for tissue regeneration according to claim 1, wherein the polymer is one or more synthetic polymers selected from a group consisting of representative bio-degradable aliphatic polyesters such as polylactic acid (PLA), polyglycolic acid (PGA), poly(D,L-lactide-co-glycolide) (PLGA), poly(caprolactone), diol/diacid aliphatic polyester, polyester-amide/polyester-urethane, poly(valerolactone), poly(hydroxyl butyrate) and poly(hydroxyl valerate) or one or more natural polymers selected from a group consisting of chitosan, chitin, alginic acid, collagen, gelatin and hyaluronic acid.

3. The fibrous porous 3-dimensional scaffold for tissue regeneration according to claim 2, wherein the polylactic acid (PLA) is a low molecular and/or a polymer poly-L-lactic acid (PLLA).

4. The fibrous porous 3-dimensional scaffold for tissue regeneration according to claim 1, wherein the fiber is 1-15 < in diameter.

5. A method for preparing the fibrous porous 3-dimensional scaffold for tissue regeneration of claim 1 by using electrospinning.

6. The method for preparing the fibrous porous 3-dimensional scaffold for tissue regeneration using electrospinning according to claim 5, which comprises the following steps:
(i) preparing a spinning solution by dissolving a polymer and/or a low-molecular compound singly or together in an organic solvent; and
(ii) spinning the polymer solution by using an electro-spinner and volatilizing the organic solvent at the same time to form a 3-dimensional network structure.

7. The method for preparing the fibrous porous 3-dimensional scaffold for tissue regeneration according to claim 5, which additionally includes the step of molding the fiber to fit defective area.

8. The method for preparing the fibrous porous 3-dimensional scaffold for tissue regeneration according to claim 5, wherein the polymer and/or low molecular compound is poly-L-lactic acid (PLLA).

9. The method for preparing the fibrous porous 3-dimensional scaffold for tissue regeneration according to claim 5, wherein the organic solvent is one or more compounds selected from a group consisting of chloroform, dichloromethane, dimethylformamide, dioxane, acetone, tetrahydrofurane, trifluoroethane and hexafluoroisopropylpropanol.

10. The method for preparing the fibrous porous 3-dimensional scaffold for tissue regeneration according to claim 9, wherein the organic solvent is a mixture of dichloromethane and propylpropanol or a mixture of dichloromethane and acetone.

11. The method for preparing the fibrous porous 3-dimensional scaffold for tissue regeneration according to claim 5, wherein the organic solvent has a boiling point of 0-40° C. and a viscosity of 25-35 cps.

12. The method for preparing the fibrous porous 3-dimensional scaffold for tissue regeneration according to claim 5, wherein the polymer and low molecular compounds are dissolved in 5-20 weight % organic solvent to prepare a spinning solution.

13. The method for preparing the fibrous porous 3-dimensional scaffold for tissue regeneration according to claim 5, wherein the step (ii) is carried out under the following conditions; temperature: 15-25° C., humidity: 10 40%, spinning distance: 10-20 cm, voltage: 10-20 kV, releasing speed: 0.050 < 0.150 ml/min and the internal diameter of the syringe: 0.5-1.2 mm.

14. An implantation material for cell adhesion, growth and regeneration comprising the fibrous porous 3-dimensional scaffold for tissue regeneration of claim 1.

15. The implantation material for cell adhesion, growth and regeneration according to claim 14, wherein the cell is cartilage cell, endothelial cell, skin cell, osteocyte, bone cell or stem cell.
